(19) United States
(12) Patent Application Publication
(10) Pub. No.: US 2024/0150697 A1
(43) Pub. Date: May 9, 2024

(54) RECONFIGURABLE BIOPROCESSING SYSTEMS

(71) Applicant: CYTIVA SWEDEN AB, Uppsala (SE)

(72) Inventors: Klaus Gebauer, Uppsala (SE); Tim Francois, Uppsala (SE); Andreas Lundin, Uppsala (SE); Bjorn Markus Olovsson, Uppsala (SE); Kerstin Erickson, Uppsala (SE); Tomas Dalmo, Uppsala (SE)

(21) Appl. No.: 18/550,075

(22) PCT Filed: Apr. 25, 2022
(86) PCT No.: PCT/EP2022/060891

§ 371 Date: Sep. 11, 2023

(30) Foreign Application Priority Data
Apr. 26, 2021 (GB) 2105923.3

(51) Int. Cl. C12M 1/00; C12M 1/36; C12M 3/00

(52) U.S. Cl. CPC C12M 23/40; C12M 23/28; C12M 23/44; C12M 29/04; C12M 29/06; C12M 41/48

(57) ABSTRACT

The present invention relates to a reconfigurable bioprocessing system (80) operable to perform a predetermined set of bioprocessing operations therein. The reconfigurable bioprocessing system (80) comprises a base unit (60) comprising a plurality of valve actuators (102). At least one of the plurality of valve actuators (102) is operable to releasably engage with at least part of a pinch valve cassette (103, 203) to control fluid flow through the pinch valve cassette (103, 203). At least part of the pinch valve cassette (103, 203) is removably attachable to the base unit (60) and the pinch valve cassette (103, 203) is provided adjacent to said plurality of valve actuators (102). The reconfigurable bioprocessing system (80) also includes a control system operable to selectively actuate respective of said plurality of valve actuators (102) to provide a valve configuration within the pinch valve cassette (103, 203) to enable the base unit (60) and the pinch valve cassette (103, 203) together to perform one of the predetermined set of bioprocessing operations. A flow kit (107) for use in reconfigurable bioprocessing systems (80) and various methods (1000) for reconfiguring bioprocessing systems (80) (e.g. automatically) are also disclosed.

### TECHNICAL FIELD

The present invention relates generally to bioprocessing systems. More particularly, the present invention relates to a reconfigurable bioprocessing system that is operable to perform various different bioprocessing operations therein of different types and/or to use different sets of process parameters.

### BACKGROUND

Various bioprocessing systems are known for performing various bioprocessing operations. For example, a liquid chromatography separation bioprocessing operation may be performed to separate desired protein, vaccine, etc., components produced in a bioreactor from other components. A commercially available product, such as an ÄKTA™ Ready Liquid Chromatography System available from Cytiva™ Lifesciences, may be used for such a purpose. Further examples of bioprocessing systems are also discussed in WO 2019/081684 A1 and EP 2 415 856 A1, for example.

Bioprocessing operations have, over the last decade or so, increasingly started to use various single use (SU) components therewith so as to reduce or eliminate the need to clean and/or sterilise any reusable components used therein. For example, single use cassettes are known for use in separating blood components. See, for example, WO 02/056992 A1 and WO 2007/136821 A1.

Certain single use components may also be provided as a part of a disposable flow kit having disposable flow paths and/or flow path elements provided therein. For example, disposable flow kits may be provided that incorporate flexible tubing manifolds that can be compressed so as to provide pinch valves for controlling fluid flow within such flow kit. U.S. Pat. Nos. 10,738,900 B2 and 8,235,067 B2 describe such single use pinch valves, for example.

However whilst bioprocessing systems provided with disposable flow kits have increased capacity and ease of use in the art, they are generally still only limited to providing one dedicated type of bioprocessing operation (e.g. chromatography, blood processing, filtration, or mixing, etc.), and may also require relatively specialised technically-trained operators in order to set them up for use.

Hence the present invention, as defined by the appended claims, is provided.

### SUMMARY OF INVENTION

Various aspects and embodiments of the present invention are defined by the appended claims.

In accordance with a first aspect, the present invention provides a reconfigurable bioprocessing system that is operable to perform a predetermined set of bioprocessing operations therein. The predetermined set of bioprocessing operations preferably includes two or more different types of bioprocessing operation such as a chromatography operation, a mixing operation and/or a filtration operation, etc.

The reconfigurable bioprocessing system includes a base unit comprising a plurality of valve actuators and a control system operable to selectively actuate respective of said plurality of valve actuators. At least one of the plurality of valve actuators is operable to releasably engage with at least part of a pinch valve cassette to control fluid flow through the pinch valve cassette, with at least part of the pinch valve cassette being removably attachable to the base unit. During operation, the pinch valve cassette is positioned adjacent to said plurality of valve actuators.

One or more components that comprise the pinch valve cassette are preferably provided as part of a single use flow kit that is used within the bioprocessing system to perform one specific bioprocessing operation. In various embodiments, a flow kit may, optionally, be further adapted to provide for at least one sensor functionality realized within such a pinch valve cassette.

The control system is then operable to selectively actuate respective of said plurality of valve actuators so as to provide a valve configuration within the pinch valve cassette to enable the base unit and the pinch valve cassette together to perform one of the predetermined set of bioprocessing operations.

By providing such a reconfigurable bioprocessing system, not only can a single base unit be used to provide multiple different types of bioprocessing operation, but also such a bioprocessing system can be operated by less skilled users/operators than are conventionally required, as will be apparent from the description given below.

Further benefits and advantages of aspects and embodiments of the present invention will also be apparent to the skilled person when reading the disclosure provided herein.

### DRAWINGS

Various aspects and embodiments of the present invention are described in more detail below with reference to the appended drawings, in which:

FIG. 1 schematically shows a base unit of a reconfigurable bioprocessing system for use in accordance with various embodiments of the present invention;

FIG. 2 shows the base unit of FIG. 1 connected to a disposable flow kit to provide a reconfigurable bioprocessing system in accordance with an embodiment of the present invention;

FIG. 3 shows a valve module arrangement unit comprising a plurality of valve actuators provided at an actuator plate for use in the base unit of FIG. 1;

FIG. 4 shows a valve module arrangement comprising a pinch valve cassette and a plurality of valve actuators provided at an actuator plate for use in a base unit of a reconfigurable bioprocessing system in accordance with an embodiment of the present invention;

FIG. 5 shows a valve module arrangement for use in a base unit of a reconfigurable bioprocessing system in accordance with various embodiments of the present invention;

FIG. 6 shows a flexible tubing manifold provided within the valve module arrangement of FIG. 5, and which flexible tubing manifold may also be provided for use with the valve module arrangement of FIG. 4;

FIG. 7 shows further valve module arrangements, including pinch valve cassettes, for use with a base unit of a reconfigurable bioprocessing system in accordance with an embodiment of the present invention;

FIGS. 8A and 8B show a first pattern of valve actuators provided at an actuator plate attached to a bioprocessing system base unit and respectively configured for use in a first and a second bioprocessing operation;

FIG. 9 shows a second pattern of valve actuators provided at an actuator plate attached to a bioprocessing system base unit and respectively configured for use in at least one bioprocessing operation;

FIG. 10 shows a valve module arrangement comprising a pinch valve cassette and a plurality of valve actuators provided at an actuator plate for use in a base unit of a reconfigurable bioprocessing system in accordance with an embodiment of the present invention;

FIG. 11 shows an exoskeleton insert for use with the pinch valve cassette of FIG. 10;

FIG. 12 shows a connecting arrangement for the exoskeleton insert of FIG. 11 in greater detail;

FIG. 13 shows the valve module arrangement of FIG. 10 in an opened state;

FIG. 14 shows a reconfigured version of the valve module arrangement of FIG. 10 in an opened state;

FIG. 15 shows schematically shows a further base unit of a reconfigurable bioprocessing system for use in accordance with various embodiments of the present invention;

FIG. 16 shows a frontal view of the base unit of FIG. 15;

FIG. 17 shows a method for reconfiguring a bioprocessing system in accordance with various embodiments of the present invention; and

FIGS. 18A and 18B show embodiments of flexible tubing manifolds for use in a reconfigurable bioprocessing system of the present invention.

### DETAILED DESCRIPTION

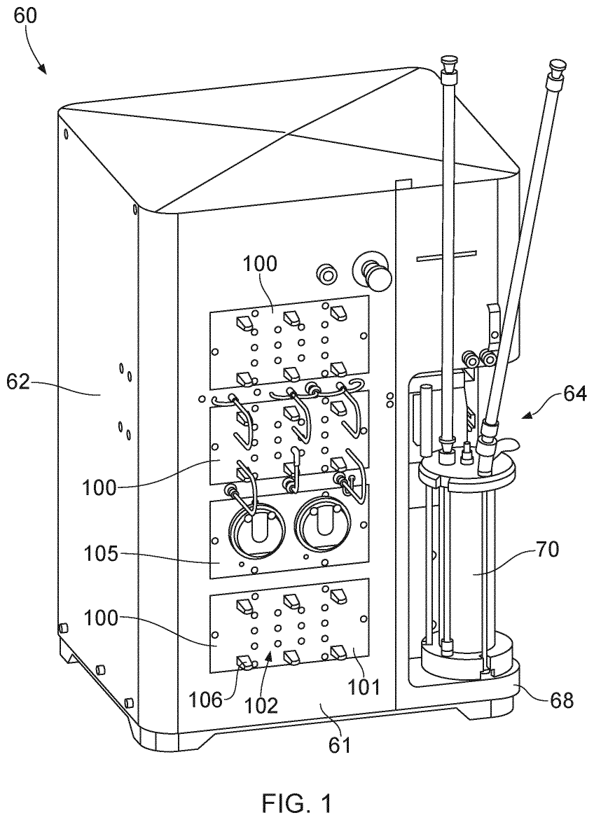

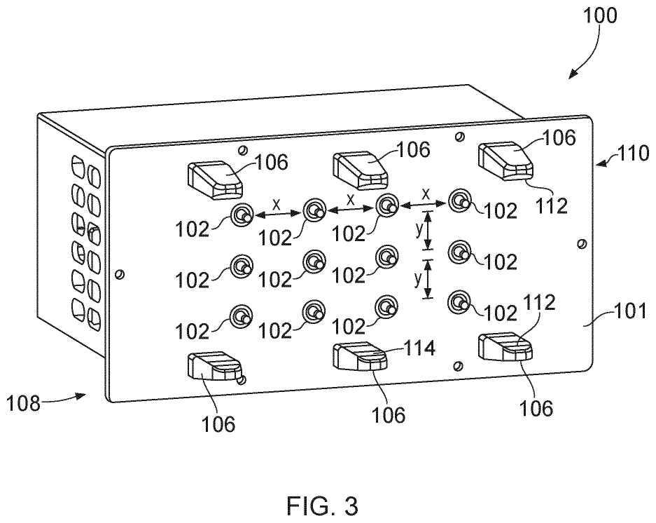

FIG. 1 schematically shows a base unit 60 of a reconfigurable bioprocessing system 80 for use in accordance with various embodiments of the present invention. The reconfigurable bioprocessing system 80 comprises the base unit 60 having a housing 62 on a front face 61 of which a plurality of valve actuators 102 are provided. Main bodies of the plurality of actuators 102 are housed within the housing 62, and the actuators 102 additionally have portions that extend outwardly through the front face 61 through a respective actuator plate 101 of a valve actuation unit 100 (see FIG. 3, for example, for more detail). Whilst three distinct valve actuation units 100 are shown in the embodiment of FIG. 1, various embodiments may be provided which use fewer valve actuation units 100, or an alternative arrangement for providing the valve actuators 102 at the front face 61 (or a different face) of the housing 62.

Housing 62 includes an elongated recess 64 which is provided at a corner thereof adjacent to the front face 61. A removable chromatography column 70 is shown as being provided in the recess 64, upon a base portion 68 coupled to or formed as part of the housing 62, and may be used to perform a bioprocessing operation involving chromatography, or variants thereof that require the use of different sets of process parameters. The chromatography column 70 is thus provided within the footprint of the housing 62 and may be used, optionally if needed, for one or more types of bioprocessing operation.

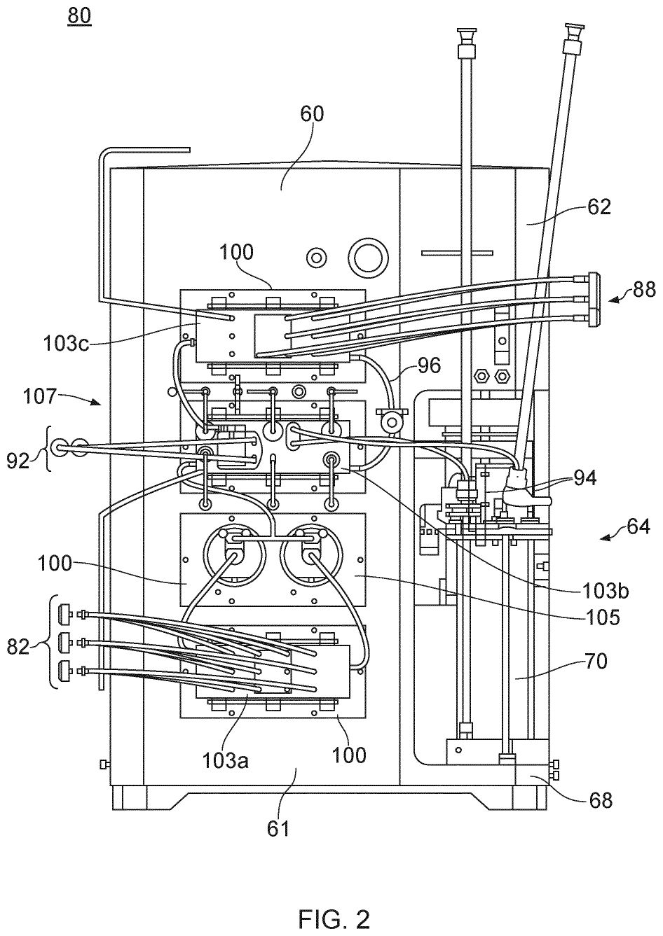

In addition to the three distinct valve actuation units 100 provided at the front face 61 of the housing 62, a pump module unit 105 is also provided within the base unit 60. This pump module unit 105 preferably includes high and low pressure fluid pumps that can be connected to a flow kit 107 of the type that is described below in connection with FIG. 2. In use these pumps can act to drive fluid flow through the flow kit 107 so as to enable a bioprocessing operation to be performed.

The valve actuation units 100 comprise a plurality of projecting resiliently moveably protrusions 106 extending substantially perpendicularly from the front face 61 (FIG. 3 shows these in more detail). Each valve actuation unit 100 may be respectively releasably coupled to a pinch valve cassette 103a, 103b, 103c of the type shown as part of the flow kit 107 of FIG. 2 using the protrusions 106, such that at least one pinch valve cassette 103a, 103b, 103c is removably attachable to the base unit 60 so as to position the pinch valve cassette 103a, 103b, 103c adjacent to the plurality of valve actuators 102.

The portions of the valve actuators 102 that extend outwardly through the front face 61 of the base unit 60 thus can project into the pinch valve cassettes 103a, 103b, 103c, once they are installed, to releasably engage flexible tubing manifolds provided therein. Activation or deactivation of the valve actuators 102 by a control system causes these outwardly extending portions to apply or release pressure on the flexible tubing manifolds to provide pinch valve activation/deactivation, which may thus in turn be used to control fluid flow through the pinch valve cassette(s) 103a, 103b, 103c.

Thus the control system is operable to selectively actuate respective of said plurality of valve actuators 102 to provide a valve configuration within the pinch valve cassette 103a, 103b, 103c to enable the base unit 60 and the pinch valve cassette(s) 103a, 103b, 103c together to perform one of a predetermined set of bioprocessing operations.

In various embodiments, such a predetermined set of bioprocessing operations may include different types of bioprocessing operations such as one or more of a chromatography operation, a mixing operation and/or a filtration operation, etc. The predetermined set may additionally, or alternatively, include operation using the same type of bioprocessing operation but accommodating different diameters of flow path conduits and/or tubing etc., thereby accommodating different operating flow rates, processing volumes and/or system hold-up volumes that may be required. The predetermined set may comprise operation of more or less complex flow paths within one type of unit operation.

However, it is to be understood that such a set of bioprocessing operations may, additionally or alternatively, include one or more different types of bioprocessing operation where the operating parameters thereof are changed. For example, a chromatography operation may be chosen and one or more different types of such a chromatography operation and/or sets of operating parameters may be selected (e.g. for high-performance liquid chromatography (HPLC), reversed-phase chromatography (RPC), size-exclusion chromatography, ion-exchange chromatography, and/or simulated moving bed (SMB) chromatography, etc.)

In other examples, unit operations other than chromatography may be chosen, such as filtration, fluid conditioning (e.g. including mixing and/or reaction steps etc.) With filtration operations, different types of filtration may be selected, such as normal flow filtration, tangential flow filtration (TFF), virus filtration, crossflow filtration with retentate recirculation (multi-pass filtration) and/or crossflow filtration in single-pass filtration configurations, etc.

FIG. 2 shows a view of the front face 61 of the base unit 60 of FIG. 1 when connected to a disposable flow kit 107 which is attached thereto in order to provide a reconfigurable bioprocessing system 80 in accordance with an embodiment of the present invention.

The flow kit 107 that is selected depends upon the bioprocessing operation that is desired. In this example, the flow kit 107 is provided for a bioprocessing operation that includes a chromatography operation. The flow kit 107 may, for example, be provided packaged in a pre-sterilised form.

In this embodiment, the flow kit 107 comprises several pinch valve cassettes 103a, 103b, 103c. The number of such pinch valve cassettes provided is however not a limiting factor. Respective of the pinch valve cassettes 103a, 103b, 103c may be formed as a “sandwich” type structure having a base cassette plate and an outer cassette plate. The base cassette plate and the outer cassette plate are coupled together and together provide at least one channel therebetween.

The base cassette plate may incorporate a plurality of vias/holes which, when the pinch valve cassette 103a, 103b, 103c is in situ, align with respective of the valve actuators 102. Such vias may thus then extend substantially perpendicularly to the surface of a respective actuator plate 101. The portions of the valve actuators 102 that extend outwardly through the front face 61 of the base unit 60 pass through respective vias and are movable within the respective channels formed between the base cassette plate and the outer cassette plate.

In certain embodiments, the valve actuators 102, or a part thereof, may act directly upon respective of the flexible tubing manifolds. The valve actuators 102 may also be used to drive an actuator head (not shown) positioned therebetween. Any such actuator head(s) may be provided as a consumable part of the flow kit 107.

At least a part of one or more of the base cassette plate and the outer cassette plate may include a relatively hard, polymer-based material. For example, such materials may include one or more of: acrylonitrile butadiene styrene (ABS), thermoplastics, polyolefins, polyethylene (PE), polypropylene (PP), polyetherimide (PEI), ULTEM resins, aliphatic polyamides, Nylon, polyphenylsulphone, RADEL, fluoropolymers, polyvinylidene fluoride (PVDF), perfluoroalkoxy (PFA), polytetrafluoroethylene (PTFE), polycarbonate, polysulphone (PSU), etc. Various other materials, such as metallic materials (e.g. stainless steel), may alternatively, or additionally, be used.

The flow kit 107 further comprises a flexible tubing manifold provided within one or more channel formed between the base cassette plate and the outer cassette plate. The flexible tubing manifold is preferably, but not necessarily, fixed between the base cassette plate and the outer cassette plate. Such a flexible tubing manifold may comprise, for example, a single straight section of tubing, but most embodiments thereof will include a plurality of interconnected conduits. These may be formed of substantially straight and/or curved sections of tubing as necessary for any particular bioprocessing operation.

The flexible tubing manifold preferably comprises at least one elastomeric material. The flexible tubing manifold may be reinforced on at least a portion thereof, within tubing of the flexible tubing manifold itself and/or externally thereto. In various embodiments, the at least one elastomeric material may include one or more of: silicone rubber; a thermoplastic elastomer (TPE); and/or a thermoplastic rubber (TPR).

Various embodiments of the invention may use single-use technology. Such embodiments may thus include one or more component(s), such as aseptic connectors, etc., that can be pre-sterilized. For example, gamma sterilisation may be used and various materials that are compatible therewith can be provided.

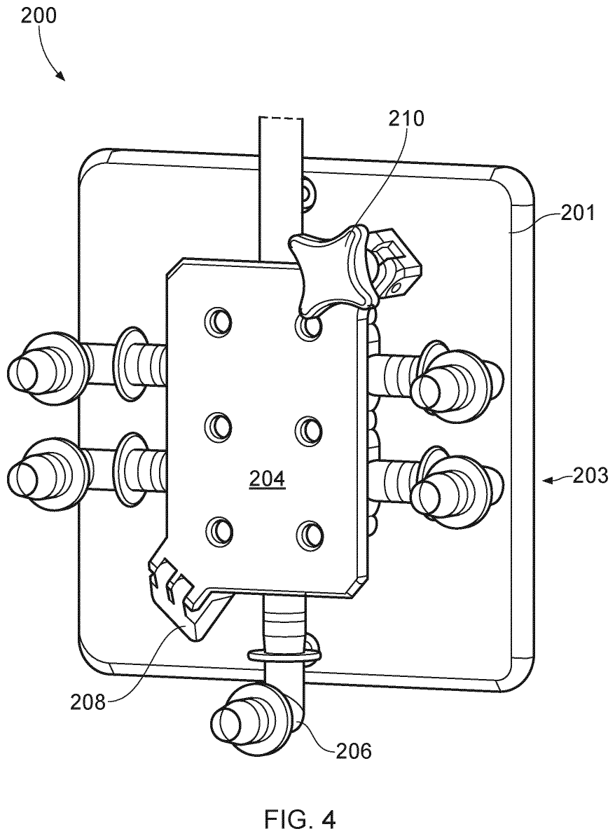

A body of the pinch valve cassette 103a, 103b, 103c, as formed by the base cassette plate and the outer cassette plate, is held in position adjacent to a respective actuator plate 101 by six projecting resiliently moveably protrusions 106 provided on a valve actuation unit 100. The protrusions 106 may retain the body of the pinch valve cassette in position by friction and/or by using hook-like portions that clamp over the outer cassette plate as the body of the pinch valve cassette 103a, 103b, 103c is pushed into an operating position adjacent the actuator plate 101. Other mechanisms for releasably attaching pinch valve cassettes 103a, 103b, 103c are also envisaged, and various such examples are described below in connection with FIGS. 4, 5, 7 and 10. The pinch valve cassette 103a, 103b, 103c is thus removably attachable into the bioprocessing system 80.

Various surface relief guide features or other guide features (not shown) may also be provided so as to ensure that the body of the pinch valve cassette 103a, 103b, 103c formed by the base cassette plate and the outer cassette plate can be easily and correctly located with respect to the actuator plate 101.

Movement of the valve actuators 102 within the channels of the pinch valve cassette 103a, 103b, 103c provides pinch valve operation therein. For example, activating a valve actuator 102 will cause an outwardly extending portion thereof to engage with part of the flexible tubing manifold within a channel of the pinch valve cassette 103a, 103b, 103c to urge it towards the outer cassette plate, thereby causing the flexible tubing manifold to close and prevent fluid flow therethrough. Deactivating the valve actuator 102 subsequently causes the flexible tubing manifold to resiliently return towards its initial open state, thus opening the flexible tubing manifold so as to enable fluid flow therethrough.

A first pinch valve cassette 103a provides fluidic communication between inlets 82 the pumps of the pump module unit 105, and acts as an inlet manifold. The pumps drive the flow of fluid within the system 80. The first pinch valve cassette 103a may be configured so that different numbers of inlets 82 may be provided to each of the pumps. The configuration of the flow through the first pinch valve cassette 103a is determined by the valve configuration provided by the selective actuation of the plurality of valve actuators 102 in the valve actuation unit 100 to which the first pinch valve cassette 103a is attached.

The pumps are also fluidically connected to a T-section 86, which allows for mixing of different fluids (where, for example, a different fluid is provided to each pump), or for increased fluid pressure (where, for example, the same fluid is provided to each pump). The outlet from the T-section 86 is fluidically connected to a second pinch valve cassette 103b.

The second pinch valve cassette 103b operates in the form of a system valve cassette, or main processing unit, which acts to direct fluid flow via a separation unit (here the chromatography column 70). This provides switchable fluid connections, via respective interconnected conduits of a flexible tubing manifold, to allow fluid to be controllably routed through (i) an air trap; (ii) a filter connected to a first pair of ports 92; and (iii) the chromatography column 70, which is connected to a second pair of ports 94. The routing of fluid through these components is determined by the valve configuration provided by the selective actuation of the plurality of valve actuators 102 in the valve actuation unit 100 to which the second pinch valve cassette 103b is attached. For example, fluid that passes through the chromatography column 70 can be returned to the second pinch valve cassette 103b via one of the second pair of ports 94.

Various other sensing devices (e.g. for process control and monitoring) may optionally also, or alternatively, be connected to the second pinch valve cassette 103b through a conduit of the flexible tubing manifold. For example, pressure sensing devices, salt level/concentration detection devices, conductivity sensing devices, UV protein detectors/flow cells, and/or air level/presence detection devices, etc. may also be provided.

An output connector 96 fluidically connects the second pinch valve cassette 103b to a third pinch valve cassette 103c. Fluid that passes through the chromatography column 70 can thus be channelled from the second pinch valve cassette 103b to the third pinch valve cassette 103c through the output connector 96. In various embodiments, a UV flow cell may be provided within the output connector 96 (or elsewhere in the bioprocessing system 80 if desired) to enable the presence of chromatographically-separated components (e.g. proteins etc.) to be detected.

Based upon the presence of chromatographically-separated components (and/or absence thereof, timings, other/alternative sensor inputs, etc.), the third pinch valve cassette 103c may then be operated to direct various bioproducts, waste products, etc. to an appropriate outlet 88 selected from a plurality of such outlets 88. These components may then be further processed to form finalised products, discarded, re-circulated, mixed, diluted, filtered, etc. depending on the desired bioprocessing operation.

Various embodiments of pinch valve cassettes 103 are envisaged. For example, pinch valve cassettes 103 may be provided that include one or more lever mechanism (e.g. akin to one or more piano key type arrangement). Such a lever mechanism(s) may be provided as an intermediate component between valve actuator(s) 102 and the flexible tubing manifold(s). The lever mechanism(s) thus enable actuation of pinch valves from outside of an actuator area and/or mechanical advantage to be provided to enhance the pinch valve actuation force.

FIG. 3 shows a valve actuation unit 100 comprising a plurality of valve actuators 102 provided at an actuator plate 101 for use in the base unit 60 of FIG. 1. In various embodiments, Carten® BPV series sanitary pinch valves (and sub-components/modified components thereof) available from Carten® Controls Inc., 604 West Johnson Avenue, Cheshire, Connecticut, CT 06410, USA, and as are further described in WO 2018/210403 A1 for example, may be used. ASCO™ Series 273 Pinch Valves (and sub-components/modified components thereof) available from Emerson Electric Co., 8000 West Florissant Avenue, P.O. Box 4100, St. Louis, MO 63136, USA may also be used.

The plurality of valve actuators 102 are provided in a pattern, which in this instance corresponds to a 4×3 rectangular grid pattern (4 vertical columns and 3 horizontal rows). The column spacing (x) and row spacing (y) are respectively equal therebetween, but differ in this instance between rows and columns so as to give a rectangular arrangement, i.e. x≠y (as opposed to a square pattern where x=y). Other pattern variants for the plurality of valve actuators 102 are however envisaged, certain of which will be further discussed below.

The valve actuation unit 100 comprises a plurality of projecting resiliently moveably protrusions 106 extending substantially perpendicularly from the valve actuator plate 101. In the example shown in FIG. 3, there are six protrusions. A first group 108 of three protrusions 106 is disposed on a first side of the pattern of valve actuators 102. A second group 110 of three protrusions 106 is disposed on a second side of the pattern of valve actuators 102, the second side being opposite to the first side.

Each protrusion 106 may include a projection 112 at its distal end which is configured to releasably engage with a pinch valve cassette 103a, 103b, 103c. In this configuration, a snap-fit attachment between opposing groups of protrusions 108, 110, with a portion of the pinch valve cassette 103a, 103b, 103c being held therebetween against the actuator plate 101 by the projections 112, is provided when the pinch valve cassette 103a, 103b, 103c is attached. The projections 112 may also each include a respective bevelled surface 114 to aid when inserting the pinch valve cassette 103a, 103b, 103c between the opposing groups of protrusions 108, 110.

In alternative variants of this embodiment, the protrusions 106 may be pivotable and/or lockable, e.g. either in groups or individually, so as to enable ease of removal of any used pinch valve cassettes 103a, 103b, 103c. Furthermore, various additional or alternative guiding/locking/locating features may also be provided as part of the pinch valve cassette(s) 103a, 103b, 103c or body/bodies thereof for use in conjunction with any of the embodiments that are herein disclosed.

The valve actuators 102 are individually operable by a control system provided in the base unit 60. In this example, the control system is provided in the valve actuation unit 100 itself in modular form within the base unit 60, but it is not necessarily so for all embodiments of the present invention and may be provided elsewhere in the base unit or remotely thereto. In various embodiments, the control system may include various ASCO™ controllers etc. that are commercially available from Emerson Electric Co., 8000 West Florissant Avenue, P.O. Box 4100, St. Louis, MO 63136, USA may also be used.

FIG. 4 shows a valve module arrangement 200 comprising a pinch valve cassette 203 and a plurality of valve actuators provided at an actuator plate 201 for use in a base unit 60 of a reconfigurable bioprocessing system 80 in accordance with various embodiments of the present invention.

The pinch valve cassette 203 comprises a lock plate 204 that is removably attachable to the base unit 60 at least at a corner portion thereof using a screw lock 210. Screw lock 210 is attached to the actuator plate 201 and may be pivoted with respect thereto. Lock plate 204 is further hingedly attached to the actuator plate 201 via a hinge 208. Surface features provided on the lock plate 204 and/or the actuator plate 201 define channels 211 into which a flexible tubing manifold 206 is inserted. The flexible tubing manifold 206 itself may comprise one conduit (e.g. with two ports provided therein for fluid input/output) or a plurality of interconnected conduits (e.g. having three or more ports provided therein for fluid input/output).

In various alternative embodiments, a part of a chassis wall of the base unit 60 may provide a functional equivalent to the actuator plate 201. However, by providing a separable actuator plate 201, a further degree of reconfigurability is provided, e.g. not only for replacement/change of pinch valve cassettes 203 and their configuration, but also so as to aid with service and maintenance of the bioprocessing system 80.

Whilst the hinge 208 may permanently attach the lock plate 204 to the actuator plate 201, in various preferred embodiments the hinge 208 comprises a releasably attachable mechanism. This enables various lock plates 204 to be attached to the actuator plate 201 in order to provide a modifiable set of channels to accommodate a variety of flexible tubing manifolds 206 therein. Various lock plates 204 may be provided with or as a part of a flow kit 107. Hence a bioprocessing system 80 is thus made further reconfigurable.

A flow kit 107 may be installed into the pinch valve cassette 203 provided on a base unit 60, which does not yet have such a flow kit 107 installed, by connecting a lock plate 204 to a hinge 208 attached to the actuator plate 201, if such isn't already present. Alternatively, a replacement lock plate 204 can be installed, for example, if a different type of bioprocessing operation is to be performed to that undertaken previously. A flexible tubing manifold 206 of the flow kit 107 may then be aligned with surface features of the actuator plate 201 which will form the channels in the pinch valve cassette 203. Optionally, clips etc. (not shown) may be used to retain the flexible tubing manifold 206 in position with respect to the actuator plate 201 whether or not surface features in the actuator plate 201 are provided to form a part of the channels.

If not already in an orientation that is not substantially perpendicular to the actuator plate 201, then the screw lock 210 is pivoted into such an orientation. The lock plate 204 is then pivoted about the hinge 208 such that it lies substantially parallel to the actuator plate 201. The surface features of the lock plate 204 and/or the actuator plate 201 thereby define channels within which the flexible tubing manifold 206 is positioned. To retain the flexible tubing manifold 206 in this position, the screw lock 210 is pivoted into an orientation that is substantially perpendicular to the actuator plate 201 and whereby a portion thereof resides in a notch 213. Tightening of a screw thread of the screw lock 210 causes it to bear onto a surface of the lock plate 204 and retains it in position during operation of the reconfigurable bioprocessing system 80.

To remove a flow kit 107, for example, once a bioprocessing operation is complete, the screw lock 210 may be released and pivoted towards a non-perpendicular orientation, whereby any portion thereof is clear of the notch 213. The lock plate 204 is then pivoted about the hinge 208 such that the flexible tubing manifold 206 is exposed. The flexible tubing manifold 206 may thereafter be removed.

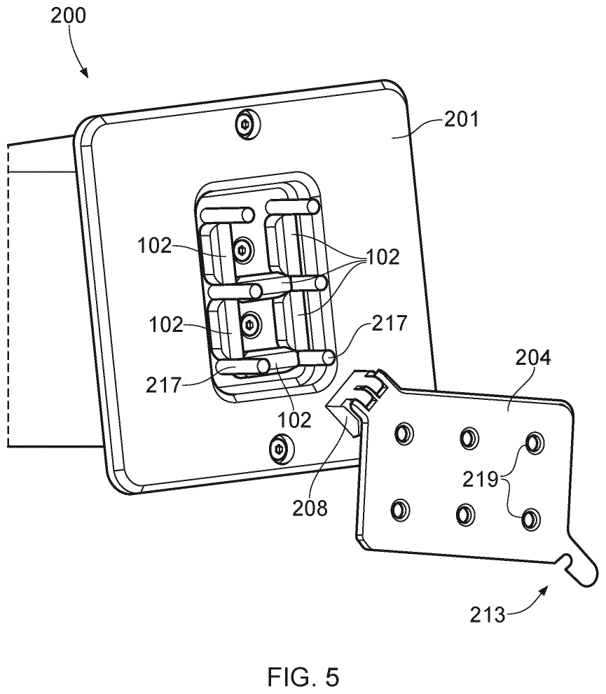

FIG. 5 shows a valve module arrangement 200 for use in a base unit 60 of a reconfigurable bioprocessing system 80 in accordance with various embodiments of the present invention. The valve module arrangement 200 of FIG. 5 shows a variant of FIG. 4 in an open state. The lock plate 204 comprises a plurality of holes 219 therein (in this case six). The valve module arrangement 200 includes (six) pins 217 that pass into respective holes 219 of the lock plate 204 as it is set into a closed position. The pins help to position/guide flexible tubing manifolds 206 into a correct position with respect to the valve actuators 102. Moreover, such an arrangement of pins 217 allows a flexible tubing manifold 206 to be held in position whilst it is substantially vertically oriented without falling or slipping downwards under the influence of gravity. Hence, an operator of the bioprocessing system does not need to hold a flexible tubing manifold 206 in place with one hand whilst simultaneously closing the valve module arrangement 200 with another hand.

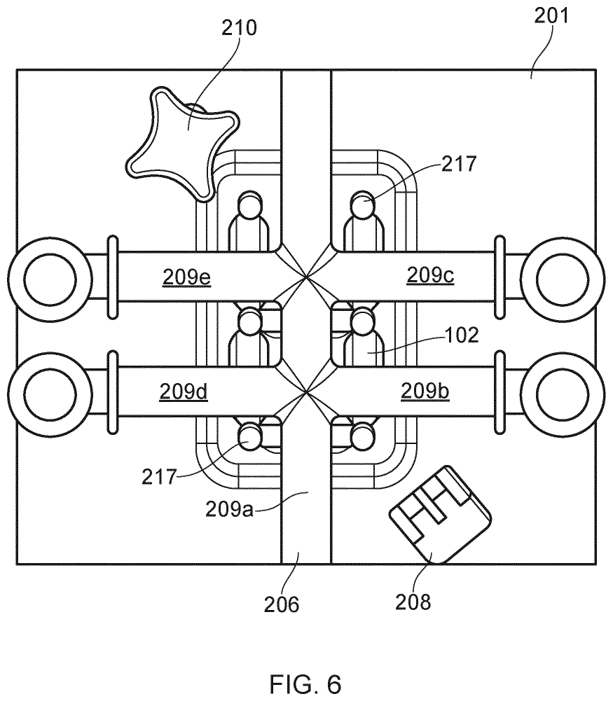

FIG. 6 shows a flexible tubing manifold 206 provided within the valve module arrangement 200 of FIG. 5. The flexible tubing manifold 206 may also be used with the valve module arrangement 200 of FIG. 4.

The flexible tubing manifold 206 is formed from a number (M) of respective conduits 209a, 209b, 209c, 209d, 209e, which is five in this case (i.e. M=5). A central conduit 209a, shown in a substantially vertical orientation, is connected to two pairs of oppositely opposed laterally extending conduits 209b, 209d, 209c, 209e. The first pair of oppositely opposed laterally extending conduits 209b, 209d is spaced with respect to the second pair of oppositely opposed laterally extending conduits 209c, 209e along a central axis of the central conduit 209a. The first pair of oppositely opposed laterally extending conduits 209b, 209d and the second pair of oppositely opposed laterally extending conduits 209c, 209e are also substantially parallel with respect to one another.

Four lowermost pins 217 support the flexible tubing manifold 206. A first lowest pair of pins 217 support the first pair of oppositely opposed laterally extending conduits 209b, 209d. A second mid-positioned pair of pins 217 support the second pair of oppositely opposed laterally extending conduits 209c, 209e. Such an arrangement thus prevents the flexible tubing manifold 206 from sliding downwards under the influence of gravity, thereby helping to facilitate flow kit installation.

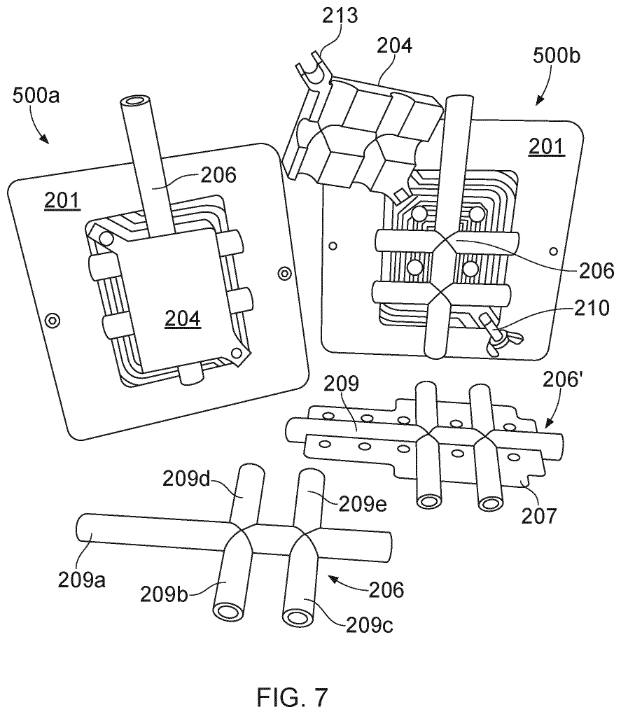

FIG. 7 shows further valve module arrangements 500a, 500b, including respective pinch valve cassettes, for use with a base unit 60 of a reconfigurable bioprocessing system 80 in accordance with various embodiments of the invention. These valve module arrangements 500a, 500b are similar to those of FIGS. 4 to 6. A first valve module arrangement 500a is shown in a closed state with a flexible tubing manifold 206 provided therein. A second valve module arrangement 500b is also depicted showing the valve module arrangement in an open state whereby the flexible tubing manifold 206 therein is exposed.

The flexible tubing manifold 206 is further depicted beneath the first valve module arrangement 500a adjacent to an alternate flexible tubing manifold 206′ that may also be used with the valve module arrangements 500a, 500b. Each of the flexible tubing manifolds 206, 206′ are formed from a number (M) of respective conduits 209a, 209b, 209c, 209d, 209e, which is five in this case (i.e. M=5). A central conduit 209a, shown in a substantially vertical orientation in the valve module arrangement 500b, is connected to two pairs of oppositely opposed laterally extending conduits 209b, 209d, 209c, 209e. The first pair of oppositely opposed laterally extending conduits 209b, 209d is spaced with respect to the second pair of oppositely opposed laterally extending conduits 209c, 209e along a central axis of the central conduit 209a. The first pair of oppositely opposed laterally extending conduits 209b, 209d and the second pair of oppositely opposed laterally extending conduits 209c, 209e are also substantially parallel with respect to one another.

Alternate flexible tubing manifold 206′ further comprises a set of six webbing portions 207 provided between respective of the conduits 209a, 209b, 209c, 209d, 209e. These are provided between: i) the central conduit 209a and the first laterally extending conduit 209b; ii) the central conduit 209a and the second laterally extending conduit 209d; iii) the central conduit 209a, the first laterally extending conduit 209b and the third laterally extending conduit 209c; iv) the central conduit 209a, the second laterally extending conduit 209d and the fourth laterally extending conduit 209e; v) the central conduit 209a and the third laterally extending conduit 209c; and vi) the central conduit 209a and the fourth laterally extending conduit 209e.

The conduits 209a, 209b, 209c, 209d, 209e may be joined by interlocking V-shaped portions thereof. These may be welded, glued, moulded, etc., and preferably provide a join area which is relatively pliable in relation to the conduits 209a, 209b, 209c, 209d, 209e away from such joins. Such join areas are thus useful as regions of activation/deformation when operating as part of a pinch valve, and they may optionally be formed using reinforced materials.

The flexible tubing manifolds 206, 206′ preferably comprise at least one elastomeric material. For example, the at least one elastomeric material may include one or more of: silicone rubber; a thermoplastic elastomer (TPE); and/or a thermoplastic rubber (TPR). The flexible tubing manifolds 206, 206′ may be reinforced on at least a portion thereof, within tubing of the flexible tubing manifolds 206, 206′ themselves and/or externally thereto. Where webbing 207, or the like, is provided this may optionally be formed using a different material from the flexible tubing manifold 206′ material. Such webbing 207 may itself be reinforced if desired (e.g. with fibres of material provided therein).

In various embodiments, one or more portions of webbing 207 may be provided with one or more holes therein through which one or more corresponding pins 217 can be placed. Such an arrangement may thus provide fixation of the flexible tubing manifold 206′ within a pinch valve cassette. Such an arrangement further facilitates a correct and precise positioning of tubing as well as increasing ease of use during installation.

Valve module arrangements 500a, 500b are similar to those shown in FIGS. 4 to 6 in so far as they comprise respective hinged lock plates 204. Optionally, the hinged lock plates 204 may be completely removable/separable from the actuator plates 201. These may be provided pre-attached to a consumable part (e.g. as part of a flow kit 107). A fitting or attachment feature may then be provided to latch the consumable part (e.g. a flow kit block) to an actuator plate 201 (e.g. preferably at actuator side thereof). In these embodiments the lock plates 204 and the actuator plates 201 are formed using a metal material, such as one or more of stainless steel, aluminium, etc., but alternative materials may be used.

The lock plates 204 comprise a plurality of channels formed therein. A notch 213 formed on the lock plates 204 is configured to engage with a movable screw lock 210, which comprises a butterfly nut, for securing the lock plates 204 to the actuator plates 201.

The flexible tubing manifolds 206, 206′ are provided adjacent to the actuator plates 201. When the lock plates 204 are in a closed position, with the movable screw lock 210 retained in the notch 213, the plurality of conduits 209a, 209b, 209c, 209d, 209e of the flexible tubing manifolds 206, 206′ are retained within respective complimentary channels of the lock plates 204. A pinch valve cassette 203 may thus be provided comprising one or more of at least a part of the actuator plate 201, a lock plate 204 and/or a flexible tubing manifold 206, 206′. One or more of such components 201, 204, 206, 206′ may further be disposable and/or provided as part of a flow kit 107.

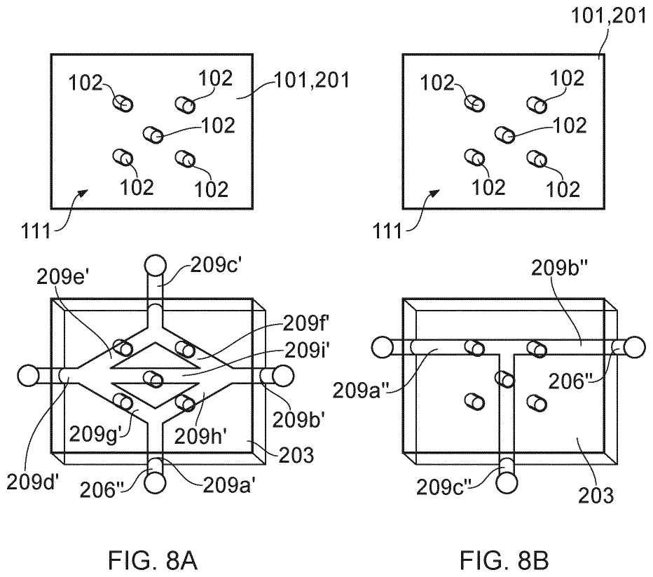

FIGS. 8A and 8B show a first pattern 111 of valve actuators 102 provided at an actuator plate 101, 201 attached to a bioprocessing system base unit 60, and respectively configured for use in a first and a second bioprocessing operation, in the upper part of each respective illustration. A number N of valve actuators 102 provided. In this instance are five valve actuators 102 are provided according to the first pattern 111 (i.e. N=5). The first pattern 111 is a quincunx.

In the lower part of FIG. 8A, a first flexible tubing manifold 206″ provided in a pinch valve cassette 203 is schematically shown in position against the actuator plate 101, 201. Lock plate 204 is not shown for reasons of clarity. The first flexible tubing manifold 206″ comprises nine respective conduits 209a′, 209b′, 209c′, 209d′, 209e′, 209f, 209g′, 209h′, 209i′. The four radially outmost conduits 209a, 209b, 209c, 209d′ are connected to respective fluid input/output ports. Four of the innermost conduits 209e, 209r, 209g, 209h′ connect adjacent of respective of the four radially outmost conduits 209a, 209b, 209c, 209d′ in a trapezoidal form. The final, fifth, innermost conduit 209i′ connects a common end of two of the innermost conduits 209e, 209g′ at a first position and a common end of the other two innermost conduits 209r, 209h′ at a second position.

A respective centre portion of the innermost conduits 209e, 209r, 209g, 209h, 209i′ is positioned adjacent a respective valve actuator 102. The fifth innermost conduit 209i′ is centrally positioned adjacent a valve actuator 102 at the centre of the quincunx of the first pattern 111. The remaining four innermost conduits 209e, 209r, 209g, 209h′ are positioned adjacent respective of the remaining valve actuators 102 at the periphery of the quincunx of the first pattern 111. Such a first flexible tubing manifold 206″ may be used, for example, to perform a chromatography type of bioprocessing operation.

In the lower part of FIG. 8B, a second flexible tubing manifold 206′″ provided in a pinch valve cassette 203 is schematically shown in position against the actuator plate 101, 201. Again, lock plate 204 is not shown for reasons of clarity. The second flexible tubing manifold 206′″ comprises three respective conduits 209a″, 209b″, 209c″ (such that M=3). The conduits 209a″, 209b″, 209c″ are connected to respective fluid input/output ports and to each other in a T-shape. However, whilst this embodiment is described as having three conduits 209a″, 209b″, 209c″ it would be understood by those skilled in the art that the colinear first and second conduits 209a″, 209b″ could be formed from a single common tubing portion.

A respective innermost portion of the conduits 209a″, 209b″, 209c″ proximal to where they connect is positioned adjacent a respective valve actuator 102. An innermost portion of the third conduit 209c″ is positioned adjacent a valve actuator 102 at the centre of the quincunx of the first pattern 111. Innermost portions of the first and second conduits 209a″, 209b″ are positioned adjacent the two uppermost valve actuators 102 of the quincunx of the first pattern 111. In this configuration two of the (N=5) valve actuators 102 are thus not used. Such a second flexible tubing manifold 206′″ and configuration may be used, for example, to perform a mixing type of bioprocessing operation.

In various embodiments, an actuator layout pattern and/or flexible tubing manifold(s) 206″, 206′″ may be provided that allow for various fluid conduit orientations to be provided therein. This allows even more re-configurability options to be enabled. For example, whilst FIG. 8A shows four conduits orientated at substantially 45 degrees to a central substantially horizontally orientated conduit, such an arrangement is not limiting. Various embodiments might thus be provided having at least one conduit orientated at various angles (e.g. >10, >20, >45, 90, from about 5 to about 45 or 40, from about 10 to 45 or 40, from about 20 to 45 or 40, from about 30 to 45 or 40, from about 5 to about 30, from about 10 to 30, from about 20 to 30, etc. degrees) with respect to the horizontal. Various embodiments may include a plurality of such conduits.

For example, an actuator may then be used to interact with a conduit oriented in a horizontal orientation for one of a multiple flow path configuration, whilst in another, second, configuration of the bioprocessing system (e.g. for another bioprocessing operation), an actuator may interact with a conduit provided in a 45 degree orientation.

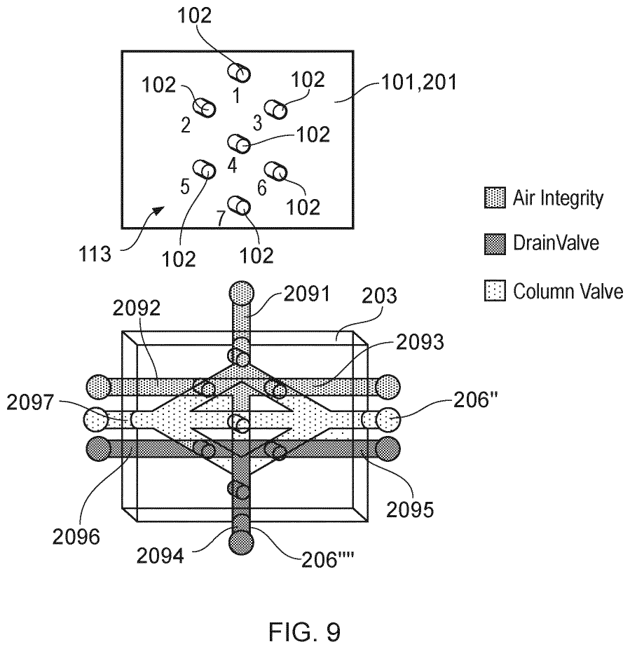

FIG. 9 shows a second pattern 113 of valve actuators 1021, 1022, 1023, 1024, 1025, 1026, 1027 provided at an actuator plate 101, 201 attached to a bioprocessing system base unit 60 and respectively configured for use in at least one bioprocessing operation in an upper part thereof. The second pattern 113 includes a centrally positioned quincunx 1022, 1023, 1024, 1025, 1026 and two additional outwardly spaced valve actuator positions 1021, 1027 that are substantially colinearly positioned with respect to a central valve actuator position 1024 of the quincunx. Hence, in this embodiment N=7. Further patterns might also be used which also include at least one quincunx layout pattern therein.

The first flexible tubing manifold 206″ of FIG. 8A is shown in phantom schematically in a pinch valve cassette 203 in the lower part of FIG. 9. Also depicted therein is a third flexible tubing manifold 206″. The third flexible tubing manifold 206″″ is similar in form to those depicted in FIGS. 6 and 7 but is formed from seven conduits 2091, 2092, 2093, 2094, 2095, 2096, 2097 (M=7). Six of the seven conduits 2091, 2092, 2093, 2094, 2095, 2096 are outwardly positioned and connected to respective fluid input/output ports. A central conduit 2097 connects respective ends of a first group of three uppermost conduits 2091, 2092, 2093 and a second group of three lowermost conduits 2094, 2095, 2096.

A centre-most valve actuator 1024 of the valve actuators 1021, 1022, 1023, 1024, 1025, 1026, 1027 is positioned adjacent to a centre portion of the central conduit 2097. The respective innermost portions of each outwardly positioned conduit 2091, 2092, 2093, 2094, 2095, 2096 are provided adjacent to a respective of the outermost valve actuators 1021, 1022, 1023, 1025, 1026, 1027 provided in the second pattern 113.

The third flexible tubing manifold 206″″ (and the flexible tubing manifold 206″ of FIG. 8A) may be used to control fluid flow to a chromatography column 70. In this embodiment, fluid flow may be directed in either a forward and reverse flow direction or alternatively or an upward or downward direction (i.e. vertically with respect to gravity) with respect to such a chromatography column 70 by use of the same single pinch valve cassette 203. Such a pinch valve cassette 203 may also be used to provide a fluid input to an air integrity test module and/or a fluid outlet or drain port in a filtration system, for example.

Similarly to FIGS. 8A and 8B above, in various embodiments, an actuator layout pattern and/or flexible tubing manifold(s) 206″, 206′″ etc. may be provided that allows for various fluid conduit orientations to be provided therein.

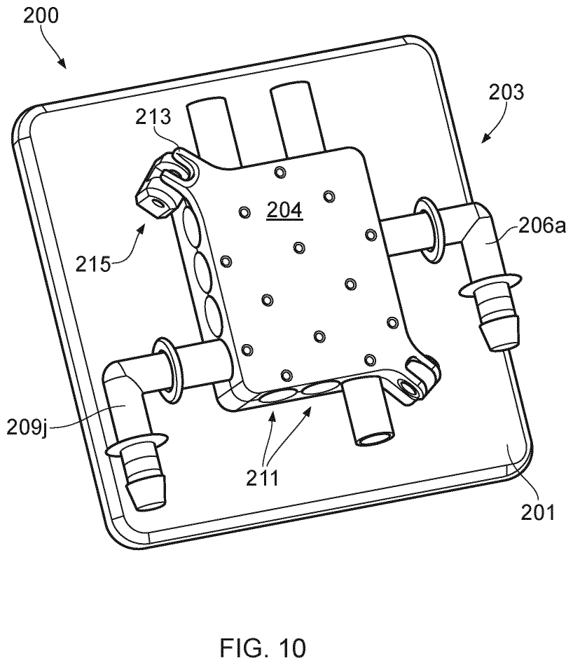

FIG. 10 shows a valve module arrangement 200 comprising a pinch valve cassette 203 and a plurality of valve actuators provided at an actuator plate 201 for use in a base unit 60 of a reconfigurable bioprocessing system 80 in accordance with an embodiment of the present invention. The pinch valve cassette 203 comprises a lock plate 204 that is hingedly attached to the base unit 60 at a corner portion thereof. In the closed position of FIG. 10, the lock plate 204 and the actuator plate 201 define a plurality of channels 211 therebetween. The actuator plate 201 and lock plate 204 may be similar to those shown in FIG. 4.

Lock plate 204 includes a notch portion 213. A notch interlock portion 215 is provided at a corner portion of the lock plate 204. Notch portion 213 is positionable within a recess of the notch interlock portion 215. Complimentary vias in both the notch portion 213 and the notch interlock portion 215 align and can be used to accommodate a locking device, such as a pin, cable tie, etc. therein.

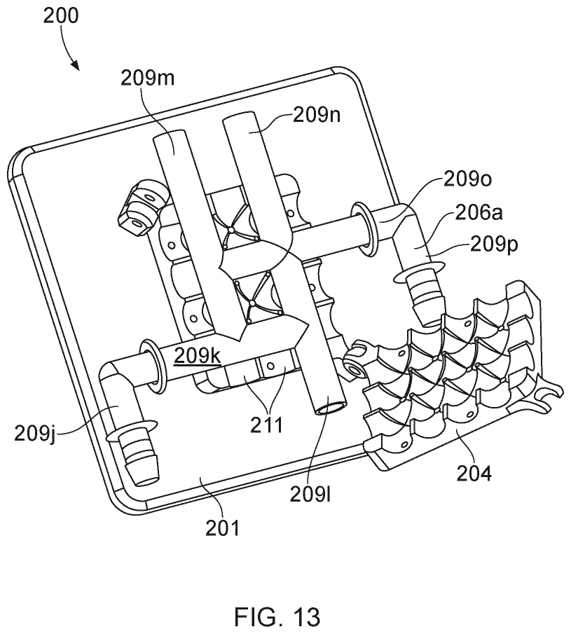

The valve module arrangement 200 also incorporates a flexible tubing manifold 206a provided in the channels 211 thereof, and which is described further in connection with FIG. 13, below.

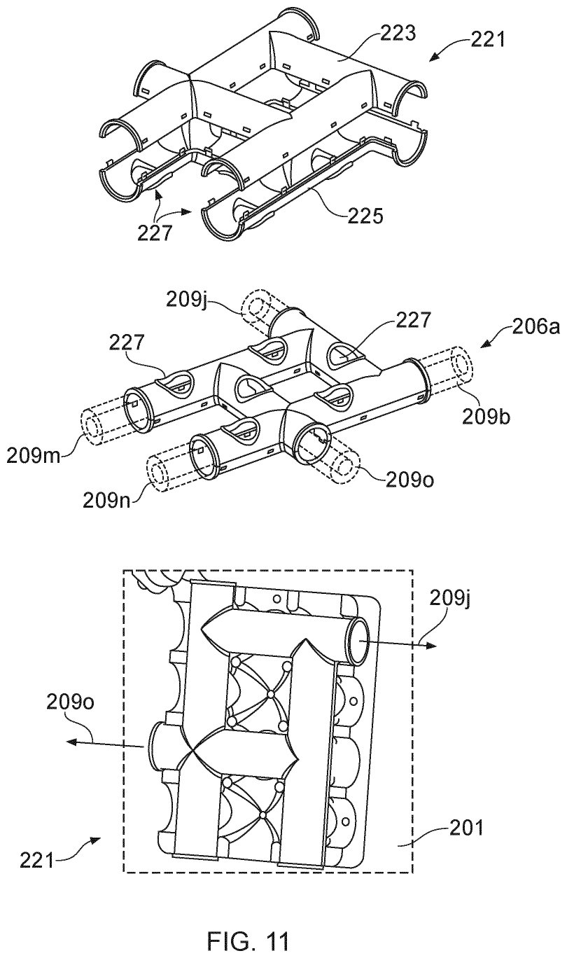

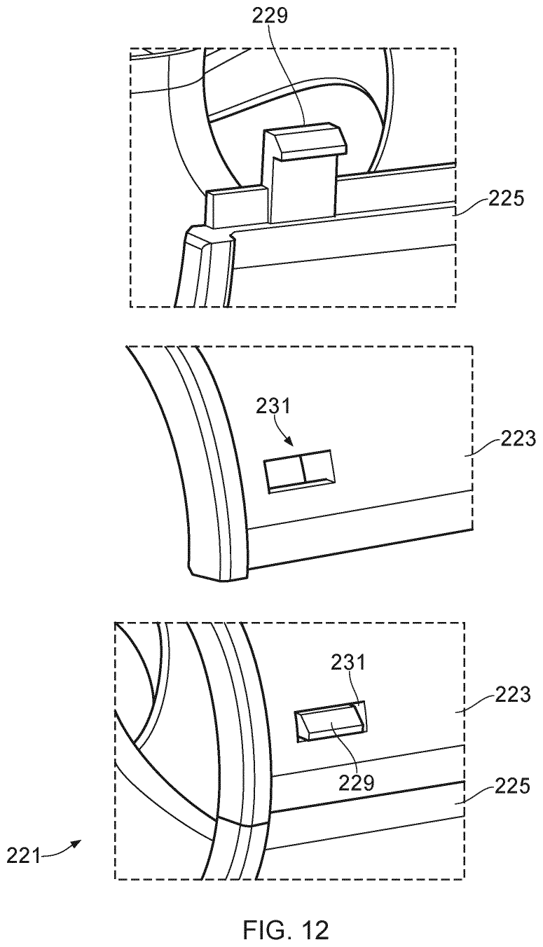

FIG. 11 shows an exoskeleton insert 221 for use with the pinch valve cassette 203 of FIG. 10. The upper illustration of FIG. 11 shows a base part 225 and a cover part 223 that together form the exoskeleton insert 221. These parts may be formed of rigid material (e.g. that is polymer-based) and clip together in a manner shown in greater detail in FIG. 12. The base part 225 comprises a set of holes 227 therein provided at positions which correspond to those of the valve actuators of the base unit 60.

The central illustration of FIG. 11 shows a flexible tubing manifold 206a provided within channels defined between the base part 225 and the cover part 223. The flexible tubing manifold 206a is shown in more detail in connection with FIG. 13. In practice, the flexible tubing manifold 206a and/or the exoskeleton insert 221 may be provided as part of a disposable flow kit 107, preferably with the exoskeleton insert 221 pre-mounted on the flexible tubing manifold 206a. In another embodiment, the exoskeleton insert 221 may be provided as an interchangeable and configurable part that is mounted to a pinch valve cassette 203 (or a part thereof), an actuator plate 201 and/or a lock plate 204 prior to installing the flexible tubing manifold 206a in the pinch valve cassette 203.

The lower illustration of FIG. 11 shows the exoskeleton insert 221 positioned against the actuator plate 201 of FIG. 10. In use, the exoskeleton insert 221 provides additional structural support for the flexible tubing manifold 206a. Use of an exoskeleton insert 221 may thus enable high pressure (HP) operation and/or allow various otherwise unsuitable tubing materials to be used.

Various embodiments of an exoskeleton insert may also be provided. For example, these may be provided so as to enable various different diameter tubing to be used (for example, ¼″ (6.4 mm) vs ⅜″ (9.5 mm) diameter tubing). Such exoskeleton inserts might thus have substantially the same external dimensions so as to fit a specific bioprocessing system 80 (e.g. in the channels 211 thereof) whilst differing in their inner channel diameters.

In other embodiments, the exoskeleton insert 221 may be formed by channels in an actuator plate 201 and/or channels in a lock plate 204. Mounting of the flexible tubing manifold 206a and closing of the pinch valve cassette 203 will then provide an exoskeleton around the tubing of the flexible tubing manifold 206a allowing for operation at high pressure (e.g. greater than 200 bar, from 250 to 300 bar, etc.), whilst ensuring a safe and reproducible fixation of the flexible tubing manifold 206a in the pinch valve cassette 203.

FIG. 12 shows a connecting arrangement for the exoskeleton insert 221 of FIG. 11 in greater detail. The base part 225 comprises a plurality of resiliently moveable clips 229 that are configured to engage with corresponding respective slots 231 provided in the cover part 223. A flexible tubing manifold 206a may thus be held adjacent the base part 225 whilst the cover part 223 is attached, in a manner that is known in the art.

Further, those skilled in the art would also be aware that various alternative ways to join/attach a base part 225 to a cover part 223 may be used as desired. For example, barbed connectors, cable/zip ties, etc. may be used.

FIG. 13 shows the valve module arrangement 200 of FIG. 10 in an opened state. Complementary surface features of the actuator plate 201 and the lock plate 204 are used to define the channels 211. However, such channels 211 may also be provided by surface features provided only on the actuator plate 201 or the lock plate 204.

The flexible tubing manifold 206a comprises a plurality of interconnected conduits 209j, 209k, 209l, 209m, 209n, 209o, 209p. Fluid port connected conduits 209j, 209p connect to respective first and second substantially horizontally-orientated conduits 209k, 209o. A first substantially vertically-orientated conduit 209m is connected to a midpoint of the first substantially horizontally-orientated conduit 209k and to an end point of the second substantially horizontally-orientated conduit 209o. A second substantially vertically-orientated conduit 209n is connected to a midpoint of the second substantially horizontally-orientated conduit 209o. A further third substantially vertically-orientated conduit 209l is connected to the same midpoint of the second substantially horizontally-orientated conduit 209o, and to an end of the first substantially horizontally-orientated conduit 209k at an opposite end thereof from the first fluid port connected conduit 209j. A substantially square-shaped fluid path is thus provided by the conduits 209j, 209k, 209l, 209m, 209n, 209o, 209p within a central region of the valve module arrangement 200.

Such a flexible tubing manifold 206a may provide a column valve with up- and down-flow, with the column (e.g. a chromatography column) being connected to the conduits 209l and 209m. Conduit 209n may thus not necessarily be used in this configuration, and can be maintained in a closed state by an associated valve actuator 102.

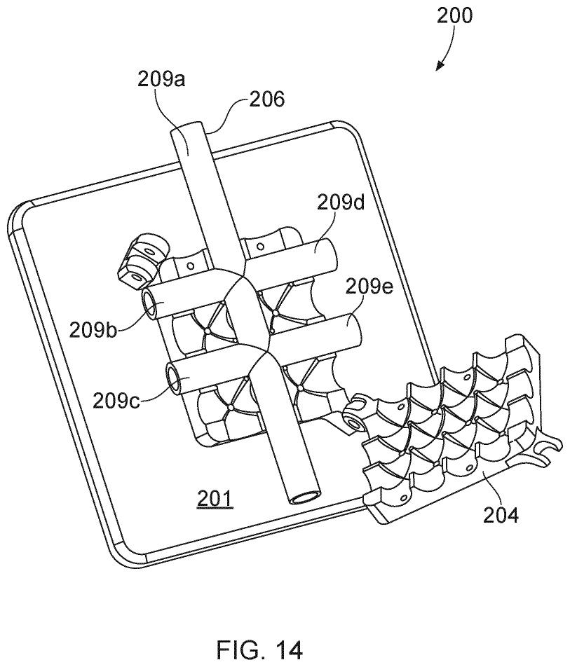

FIG. 14 shows a reconfigured version of the valve module arrangement 200 of FIGS. 10 and 13 in an opened state. An alternative flexible tubing manifold 206 is provided therein. In this instance, the flexible tubing manifold 206 is the same as is described above in relation to FIG. 7. Use of different flexible tubing manifolds 206, 206a thus enables the bioprocessing system 80 to be reconfigured so as to, for example, provide different types, sub-types, type-variants and/or to use different sets of process parameters, etc.

In this arrangement, a first (left) path may be provided by the conduits 209b, 209c, a second (right) path may be provided by the conduits 209d, 209e, a third (upper straight) path may be provided by the conduits 209b, 209d and a fourth (lower straight) path may be provided by the conduits 209c, 209e. These may be connected to various elements, such as filters, air traps, sensors, etc.

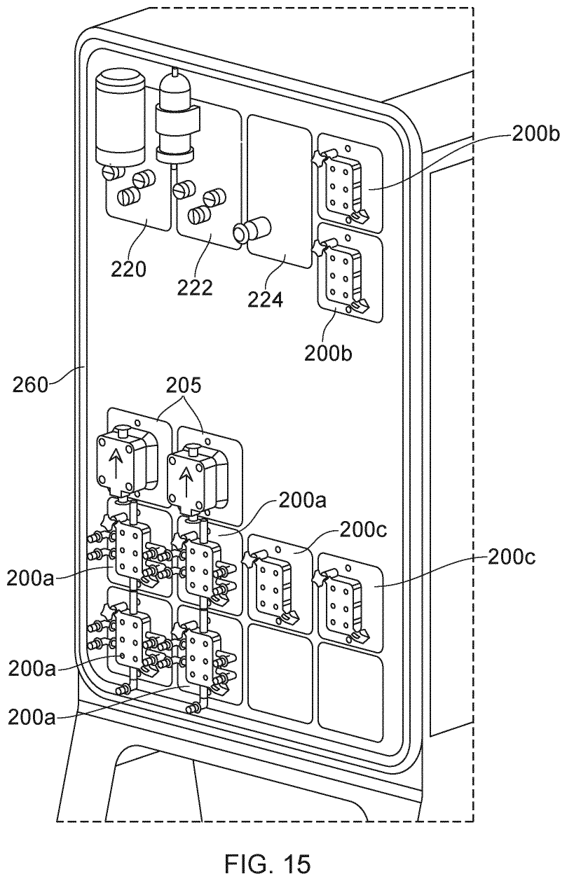

FIG. 15 shows schematically shows a further base unit 260 of a reconfigurable bioprocessing system for use in accordance with various embodiments of the present invention. The base unit includes a plurality of valve module arrangements 200 which may be of the type as described above in connection with FIGS. 4 and 5.

For example, valve module arrangements 200a may serve as inlet valve modules, valve module arrangements 200b may serve as outlet valve modules and valve module arrangements 200c may serve as column valve modules. A pair of pump module units 205 are also provided therein. An inline/bypass air trap module 220 and an inline/bypass filter 222 module are also shown schematically, but without showing details of related valve module arrangements. Module 224 shows a cover plate provided over an unused module position, the cover plate solely providing a tube holder functionality.

Base unit 260 suitably comprises further modules that are not shown in FIG. 15, such as sensor modules. Sensor modules may comprise one or several sensors, for example sensors for sensing pH, UV, conductivity, flow, air etc. The modules illustrated at a front face of the base unit 260 may be optionally provided, and their position and configuration may be changed depending on the configuration needs of the operation and the flow kit 107.

In another embodiment, one or more functional modules may be configured for permanent integration with the chassis of the base unit 260. For example, a first pump and/or a first inlet and/or outlet valve module may be provided in a fixed installation with the base unit 260, while other modules may be provided as optional modules that may be installed depending on the specific instrument and flow kit configuration desired.

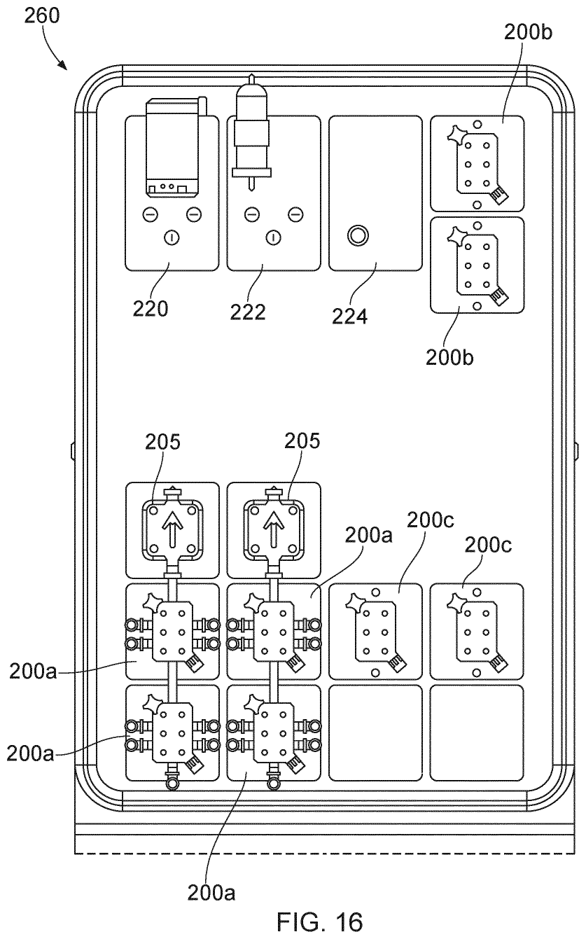

FIG. 16 shows a frontal view of the base unit 260 of FIG. 15. A control system may be provided within the base unit 260 itself, or one or more (or all) components thereof may be remotely provided. In use, the valve module arrangements 200 are easily accessible and useable. Such valve module arrangements 200 further enable quick and easy reconfiguration of a bioprocessing system 80 to be undertaken, even by non-expert users.

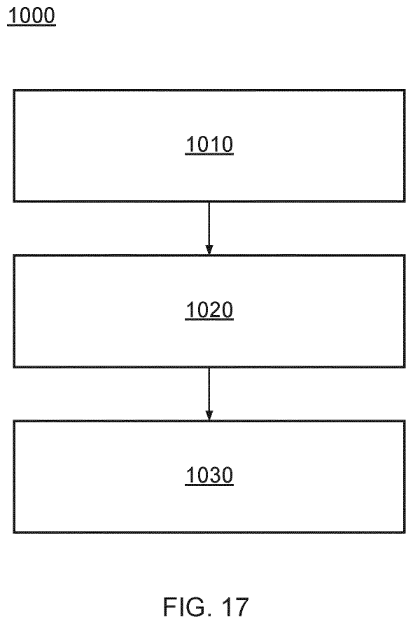

FIG. 17 shows a method 1000 for reconfiguring a bioprocessing system in accordance with various embodiments of the present invention. Such a method 1000 may be implemented by the control system of various embodiments of the present invention.

The method 1000 comprises a first step 1010 of replacing a first flow kit 107 in a bioprocessing system 80 with a second flow kit 107 for performing a bioprocessing operation that is different from that associated with said first flow kit 107. For example, a first flow kit 107 may be used to provide a mixing operation of various fluid components that are subsequently intended to be used in a chromatography separation process. A second flow kit 107 may then be used to perform the chromatography operation using the mixed fluid components. The method 1000 and first step 1010 may comprise the re-configuration of valve modules, for example by modifying or interchanging a valve lock, a valve plate, an exoskeleton not provided pre-installed with a flow kit etc. Further, method 1000 and first step 1010 may comprise a change in configuration or orientation of modules, including valve modules, within the base unit 260.

The method 1000 comprises a second step 1020 of identifying a model and/or type of the second flow kit 107. This step 1020 may include providing a graphical user interface (GUI) on a screen of a base unit 60, 260. Such a screen may be touch sensitive and allow a user to identify the type and/or model of the second flow kit 107. Such an identification allows the base unit 60, 260 to program the control system therein to control, inter alia, the valve actuators 102 in order to provide appropriate fluid flow control within the second flow kit 107 to achieve a desired bioprocessing operation.

Whilst manual identification is possible, in preferred embodiments of the present invention automatic identification of a pinch valve cassette 103a, 103b, 103c, 203, or a part thereof, is provided. Such a pinch valve cassette 103a, 103b, 103c, 203 can be automatically identified when it is provided for use in a bioprocessing system 80. The pinch valve cassette 103a, 103b, 103c, 203, a part thereof, or any other part of the flow kit 107, may thus be provided with a radio-frequency identification (RFID) tag, a 2D bar code, a near-field communication tag, an optically readable tag etc. that, for example, encodes a unique identifier (UID). The base unit 60, 260 (e.g. at each of the valve actuation units 100, valve module arrangements 200 thereof) may thus be configured to read the UID from the pinch valve cassette 103a, 103b, 103c, 203, or a part thereof, as it is installed for use, for example.

The base unit 60, 260 may be connected to a communications network. For example, a secure internet link may be provided that connects the base unit 60, 260 to a secure remotely hosted central database. A record in the central database may thus be created for each base unit 60, 260 and/or each flow kit that is sold/provided. These records can be used to identify if a particular flow kit 107 presented to a particular base unit 60, 260 is compatible therewith, is a genuine part, has already been used (and thus possibly faked), has been sterilised correctly, is subject to a product recall, is out of date, etc. Use of a specific flow kit 107 with a base unit 60, 260 may thus be inhibited as appropriate thereby e.g. so as to prevent any contaminated bioproducts being produced.

In various embodiments, the base unit 60, 260 may also provide process data and/or control system data to the central database to enable data analytics to be undertaken to provide diagnostics etc. for the base unit 60, 260 itself and/or any flow kits used therewith. Faults with a base unit 60, 260 may also be reported, e.g. thereby enabling servicing personnel to be alerted.

The method 1000 also comprises a third step 1030 of reconfiguring the bioprocessing system 80 based upon the model/type identification information of the second flow kit 107. For example, a software component provided for configuring the control unit may be programmed to change flow kit operating parameters from those associated with the first flow kit to parameters associated with the second flow kit automatically in accordance with a UID, for example. Such parameters may include timing sequences for operating each of the plurality of valve actuators 102 provided in one or more valve actuation units 100 or valve module arrangements 200, 500. Such parameters may optionally define process parameters for a specific type of bioprocessing operation (e.g. a liquid chromatography operation) but which are variable between the first and second flow kits.

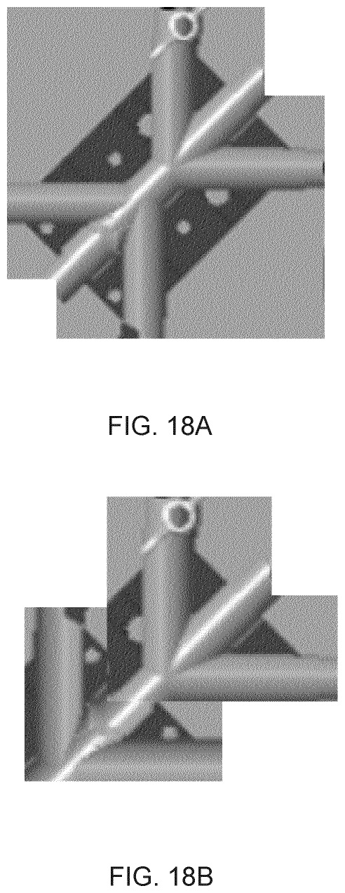

FIGS. 18A and 18B show embodiments of flexible tubing manifolds for use in a reconfigurable bioprocessing system of the present invention.

FIG. 18A shows a flexible tubing manifold having an optimized “jumping Jack”, asterisk or star shape. In this embodiment a substantially linear central conduit is provided. At one proximal end thereof two further conduits are joined to the central conduit. These are attached at an angle of substantially 45° with respect to a central axis of the central conduit in a direction facing towards a distal end of the central conduit. These two conduits thus provide respective fluid flow paths that converge on the central axis at the proximal end. Two additional conduits are also joined to the central conduit proximal to the distal end thereof. The two additional conduits are attached at an angle of substantially 45° with respect to the central axis of the central conduit in a direction facing away from the distal end of the central conduit. The two additional conduits thus provide respective fluid flow paths that diverge away from the central conduit at the distal end.

FIG. 18B shows a flexible tubing manifold having an alternative shape. In this embodiment a substantially linear central conduit is provided. At one proximal end thereof two further conduits are joined to the central conduit. These are attached at an angle of substantially 45° with respect to a central axis of the central conduit in a direction facing towards a distal end of the central conduit. These two conduits thus provide respective fluid flow paths that converge on the central axis at the proximal end. Two additional conduits are also joined to the central conduit proximal to the distal end thereof. The two additional conduits are also attached at an angle of substantially 45° with respect to the central axis of the central conduit and are substantially parallel with respective of the two further conduits.

In various embodiments, the flexible tubing manifolds of FIGS. 18A and/or 18B can be optimised such that any dead-legs therein can be minimised or substantially eliminated. Preferably, such an arrangement may be provided wherein there is no dead-leg present at all. Matching exoskeletons for such flexible tubing manifolds may also be provided.

In various embodiments of the present invention, pinch valve cassettes may also be provided with flexible tubing manifolds that are not completely substantially coplanar in their layout. For example, various 3-dimensional types of flexible tubing manifold are envisaged by the inventors. Such flexible tubing manifolds may, for example, be provided in one or more layers.

In this regard, whilst a reconfigurable bioprocessing system might be provided with different length valve actuators, for example, additionally (or alternatively) one or more intermediate actuator depth adapter may be provided for use with (or as part of) a pinch valve cassette.

Such an intermediate actuator depth adapter may be provided as part of an exoskeleton arrangement that can be used to alter the effective depth(s) within a pinch valve cassette at which actuators pinch the flexible tubing manifolds. With such an arrangement a reconfigurable bioprocessing system can be provided with a standard actuator array, whilst different consumable pinch valve cassettes can then be used according to customer need.

In various embodiments, actuators or parts thereof may be provided according to various patterns. Where such patterns include non-rectangular/square array shapes (e.g. quincunx, staggered, hexagonal, triangular, etc.) they can be made relatively compact in design/layout.

Various embodiments of the present invention may be provided in which integration of additional components and/or functionality may be provided (e.g. within a valve module arrangement, pinch valve cassette, etc.). For example, sensing of fluid and/or non-fluid parameters, identification of mounted consumables parts, and/or indicator/display features, etc. may be provided. For example, LEDs, LCDs and/or e-INK displays may be integrated into a valve module arrangement, pinch valve cassette, etc., and can be used to illustrate/display status, information relating to a flexible tubing manifold, valve positions, system parameters, fluid flow and/or sensing features etc. Such additional components and/or functionality may be activated automatically, for example, when a lid, lock plate, etc. is closed to retain part of a flow kit in situ.

Where sensing capability/function is provided it may not require any specific adaption of a consumable part. For example, various sensors may be able to work with a tubing wall material as is. For other sensors, a specific sensor window or probe section may be incorporated into a tubing manifold. Optical sensors (e.g. for air detection), proximity sensors (e.g. to detect whether a consumable part has been installed or not), ultrasonic sensors (e.g. “clamp on” used for air detection and/or fluid flow), pressure sensors (e.g. for a load cell adjacent to tubing). Sensors with electrical, optical and/or other contact points may be integrated, and the closing of a lid, lock plate, etc. simultaneously secures their mounting, fitting, and connectivity. UV sensing may be included as a lid, lock plate, etc. can itself provide protection from stray light. A conductivity probe may also be contacted when attaching a consumable part.

Various aspects and embodiments of the present invention thus provide a reconfigurable bioprocessing system that is flexible as well as being easy to use and configure. Such a reconfigurable bioprocessing system may thus reduce the need for skilled/expert/specialised operators as well as being faster and more reliable to use and reconfigure. Advantageous reconfigurability may thus be provided, for example, by virtue of providing different pinch valve cassettes where at least some (or all) of these have fewer pinch valves (e.g. a number P) therein than actuators (N) provided on a base unit of a reconfigurable bioprocessing system (i.e. N>P, where P might, optionally, also be the same as the number (M) of conduits).

Pinch valve cassettes according to various different embodiments have been described herein. The whole or any part of such pinch valve cassettes may be removably couplable into a reconfigurable bioprocessing system. For example, a pinch valve cassette may be provided having a base layer (e.g. an actuator plate, base plate, base cassette plate etc.) and a cover layer (e.g. a lock plate, outer cassette plate, cover plate, etc.) with an intermediate layer being provided therebetween in a “sandwich” type arrangement.

In various embodiments the whole “sandwich” type arrangement may be removable from a base unit of a reconfigurable bioprocessing system. In other embodiments, one or more of the base layer and the cover layer may be provided as a part of, or connected to, the base unit. Such base and/or cover layers may be movable and/or removable with respect to the base unit. An intermediate layer, for example, comprising a flexible tubing manifold may be provided. Such an intermediate layer may be releasably attachable into a pinch valve cassette, e. g. either separately from, or integral with, one or more of the base layer and the cover layer.

Various aspects and embodiments of the present invention have been described herein. However, the present invention is not to be seen as being limited by the embodiments described above, but can be varied within the scope of the appended claims as will be readily apparent to the person skilled in the art. Moreover, as far as is possible, all of the references acknowledged herein (e.g. patent applications, products/third party products, etc.) are hereby incorporated herein by reference to the maximum permissible extent.

1. A reconfigurable bioprocessing system operable to perform a predetermined set of bioprocessing operations therein, the reconfigurable bioprocessing system comprising:
a base unit comprising a plurality of valve actuators, wherein at least one of the plurality of valve actuators is operable to releasably engage with at least part of a pinch valve cassette to control fluid flow through the pinch valve cassette, and wherein at least part of the pinch valve cassette is removably attachable to the base unit; and
a control system operable to selectively actuate respective of said plurality of valve actuators to provide a valve configuration within the pinch valve cassette to enable the base unit and the pinch valve cassette together to perform one of the predetermined set of bioprocessing operations.

2. The bioprocessing system of claim 1, wherein the plurality of valve actuators are provided at an actuator plate attached to the bioprocessing system base unit in a pattern.

3. The bioprocessing system of claim 1, wherein the pattern includes at least one non-rectangular or non-square arrangement of valve actuators.

4. The bioprocessing system of claim 3, wherein said arrangement of valve actuators includes at least one hexagonal, quincunx and/or triangular actuator arrangement.

5. The bioprocessing system of any of the claim 1, wherein the predetermined set of bioprocessing operations comprises one or more of: a chromatography operation, a mixing operation and/or a filtration operation.

6. The bioprocessing system of claim 1, wherein the plurality of valve actuators are operable as pinch valve actuators in conjunction with the pinch valve cassette.

7. The bioprocessing system of claim 1, wherein the pinch valve cassette comprises a flexible tubing manifold comprising a conduit or a plurality of interconnected conduits.

8. The bioprocessing system of claim 7, wherein the flexible tubing manifold comprises at least one elastomeric material and/or is optionally reinforced.

9. The bioprocessing system of claim 8, wherein said at least one elastomeric material includes one or more of: silicone rubber; a thermoplastic elastomer (TPE); and/or a thermoplastic rubber (TPR).

10. The bioprocessing system of claim 1, wherein the pinch valve cassette comprises a lock plate that is removably attachable to the base unit.

11. The bioprocessing system of claim 1, further operable to automatically identify said pinch valve cassette when at least said removable part thereof is attached to said base unit for use.

12. The bioprocessing system of claim 11, further operable to automatically configure said base unit to perform a specific bioprocessing operation, selected from said predetermined set of bioprocessing operations, in connection with said pinch valve cassette in dependence upon said automatic identification of said pinch valve cassette or said removable part thereof.

13. A flow kit comprising at least a flexible tubing manifold for use in a pinch valve cassette, said flow kit being removably attachable to the bioprocessing system of claim 1.

14. The flow kit of claim 13, further comprising at least one of: i) at least one flexible tubing manifold comprising a plurality of interconnected conduits, wherein the at least one flexible tubing manifold is configured to be removably housed within one or more channels defined by an actuator plate and/or a lock plate; ii) an actuator plate; iii) a lock plate; iv) an intermediate actuator depth adapter; and/or v) an exoskeleton insert for housing said at least a flexible tubing manifold.

15. The flow kit as claimed in claim 13, wherein the at least one flexible tubing manifold comprises webbing provided between at least two interconnected conduits of a plurality of interconnected conduits and/or wherein the at least one flexible tubing manifold is substantially “jumping Jack”, asterisk or star shaped.

16. A method for reconfiguring a bioprocessing system, comprising:
replacing a first flow kit in the bioprocessing system with a second flow kit for performing a bioprocessing operation that is different from that associated with said first flow kit;
identifying a model/type of the second flow kit; and
reconfiguring; the bioprocessing system based upon the model/type identification information of the second flow kit.

17. The method of claim 16, comprising identifying a model/type of the second flow kit by automatically identifying at least part of a pinch valve cassette when it is provided for said bioprocessing system.
